(19) United States
(12) Patent Application Publication
(10) Pub. No.: US 2010/0112030 A1
(43) Pub. Date: May 6, 2010

(54) ROLE OF HEDGEHOG SIGNALING IN ATHEROSCLEROSIS AND CARDIOVASCULAR DISEASE

(75) Inventors: Farhad Parhami, Los Angeles, CA (US); Michael E. Jung, Los Angeles, CA (US); Khanhlinh Nguyen, Los Angeles, CA (US); Dongwon Yoo, Los Angeles, CA (US)

Correspondence Address: VENABLE LLP, P.O. BOX 34385, WASHINGTON, DC, 20043-9998, US

(73) Assignee: The Regents of the University of California, Oakland, CA (US)

(21) Appl. No.: 12/531,608

(22) PCT Filed: Mar. 17, 2008
(86) PCT No.: PCT/US08/03493

§ 371 Date: Sep. 16, 2009

Related U.S. Application Data
(60) Provisional application No. 60/907,001, filed on Mar. 16, 2007.

(51) Int. Cl. A61F 2/00; A61K 38/16; A61P 9/00; A61P 9/10; C12N 5/02; C12N 5/071; A61K 31/56; C07J 9/00

(52) U.S. Cl. 424/423; 514/12; 435/375; 435/366; 514/170; 552/544

(57) ABSTRACT

The present invention provides methods and related compositions for treating or preventing cardiovascular diseases, including, e.g., atherosclerosis, using an oxysterol. In addition, the present invention provides novel methods and related compositions for treating or preventing cardiovascular diseases, including, e.g., atherosclerosis, using a hedgehog protein, or a biologically active fragment or variant thereof.

### STATEMENT OF GOVERNMENT INTEREST

Aspects of the invention were made with U.S. government support provided by NIH/NIAMS grant number R01-AR050426. The government has certain rights in the invention.

### BACKGROUND

Cardiovascular disease is a major health risk throughout the world. Atherosclerosis, the most prevalent of cardiovascular diseases, is the principal cause of heart attack, stroke, and gangrene of the extremities, and as further associated with other vascular conditions such as cerebrovascular disease, peripheral vascular disease, stenosis, restenosis and/or in-stent-stenosis. Atherosclerosis and its hallmark feature, vascular calcification, result generally from chronic inflammatory processes that are mediated by a variety of factors, including oxidized lipids, oxidized lipoproteins, cytokines, and chemokines, all of which interact with the immune and vascular cells to mediate atherosclerotic lesion formation. Atherosclerosis is a complex disease involving many cell types and molecular factors, though a number of signaling molecules have been shown to regulate the inflammatory process in the artery wall, such as various kinases, phosphatases, receptors, and transcription factors, all of which are involved in an intricate and complex cross-talk that determines the pro- and anti-inflammatory states in the vessel wall.

Hedgehog molecules have been shown to play key roles in a variety of processes, including tissue patterning, mitogenesis, morphogenesis, cellular differentiation and embryonic development. In addition to its role in embryonic development, hedgehog signaling plays a crucial role in postnatal development and maintenance of tissue/organ integrity and function. Studies using genetically engineered mice have demonstrated that hedgehog signaling is important during skeletogenesis as well as in the development of osteoblasts in vitro and in vivo. In addition to playing a pro-osteogenic role, hedgehog signaling also inhibits adipogenesis when applied to pluripotent mesenchymal cells, C3H-10T 1/2.

Hedgehog signaling involves a very complex network of signaling molecules that includes plasma membrane proteins, kinases, phosphatases, and factors that facilitate the shuffling and distribution of hedgehog molecules. Production of hedgehog molecules from a subset of producing/signaling cells involves its synthesis, autoprocessing and lipid modification. Lipid modification of hedgehog, which appears to be essential for its functionality, involves the addition of a cholesterol molecule to the C-terminal domain of the auto-cleaved hedgehog molecule and palmitoylation at its N-terminal domain. Additional accessory factors help shuttle hedgehog molecules to the plasma membrane of the signaling cells, release them into the extracellular environment, and transport them to the responding cells.

### SUMMARY

The present invention relates generally to methods of modulating hedgehog signaling in target cells involved in the cardiovascular diseases. Certain aspects of the invention are directed to the treatment or inhibition of the pathogenesis of atherosclerosis and/or vascular calcification. In certain aspects, the target cells involved in cardiovascular disease include, for example, the cells of the arterial wall and/or other vascular or immune cells involved in the pathogenesis of atherosclerosis. Accordingly, embodiments of the present invention encompass the use of various agents, such as oxysterols and/or recombinant hedgehog proteins, to modulate hedgehog signaling in target cells involved in the pathogenesis of cardiovascular diseases, such as cardiovascular diseases associated with atherosclerosis and/or vascular calcification.

Certain embodiments provided herein include methods of treating a cardiovascular disease, comprising administering to a subject having or at risk for having the cardiovascular disease a therapeutically effective amount of a composition comprising an agent selected from a recombinant hedgehog protein and an oxysterol, thereby treating the cardiovascular disease. In certain embodiments, the cardiovascular disease is associated with, related to, or caused by, atherosclerosis and/or vascular calcification. In certain embodiments, the recombinant hedgehog protein is selected from Sonic hedgehog (Shh), Indian hedgehog (Ihh) and Desert hedgehog (Dhh) proteins, in addition to biologically active fragments or variants thereof.

According to certain methods provided herein, the oxysterol may include a naturally occurring oxysterol and/or a synthetic oxysterol. In certain aspects, a naturally occurring oxysterol may be selected from 22(S)-hydroxycholesterol, 22(R)-hydroxycholesterol, 20(S)-hydroxycholesterol, 5-cholesten-3beta, 20alpha-diol 3-acetate, 24-hydroxycholesterol, 24(S), 25-epoxycholesterol, pregnenolone, 26-hydroxycholesterol, and 4beta-hydroxycholesterol; and/or a synthetic oxysterol may be selected from a compound represented by Formula I, as detailed herein, such as Oxy1, Oxy2, Oxy3, Oxy4, Oxy5, Oxy6, Oxy7, Oxy8, Oxy9, Oxy10, Oxy11, Oxy12, Oxy13, Oxy14, Oxy15, and Oxy16, as also detailed herein.

In other embodiments, a composition employed by the methods provided herein comprises a combination of one or more recombinant hedgehog proteins, biologically active fragments or variants of a recombinant hedgehog protein, and/or various oxysterols.

In certain embodiments, the cardiovascular disease or vascular disease may be selected from any disease associated with or related to atherosclerosis and/or vascular calcification, and may also be selected from any disease associated with macrophage activation, including, but not limited to, aneurysms, angina, arrhythmia, cardiomyopathy (e.g., alcoholic, ischemic, valvular, inflammatory), stroke, cerebrovascular disease, chronic inflammatory diseases (e.g., rheumatoid arthritis, osteoarthritis, inflammatory lung disease, inflammatory bowel disease, psoriasis), congenital heart disease, congestive heart failure, coronary artery disease, myocarditis, valve disease, dilated cardiomyopathy, diastolic dysfunction, endocarditis, gangrene, hypertension, hypertrophic cardiomyopathy, ischemic heart disease, inflammatory heart disease, macrophage activation syndrome, mitral valve prolapse, myocarditis, myocardial infarction (heart attack), venous thromboembolism, peripheral artery occlusive disease, stenosis, and restenosis.

Additional embodiments include methods for treating atherosclerosis, such as by regressing or decreasing the formation of arterial atherosclerotic lesions, comprising administering to a subject having or at risk for having atherosclerosis or arterial atherosclerotic lesions a therapeutically effective amount of a composition comprising an agent selected from a recombinant hedgehog protein and an oxysterol, thereby treating atherosclerosis or regressing or decreasing formation of arterial atherosclerotic lesions.

Certain embodiments encompass methods of reducing the pathogenesis of atherosclerosis and/or vascular calcification, comprising contacting vascular or immune cells with an agent selected from a recombinant hedgehog protein and an oxysterol in an amount effective to modulate hedgehog signaling in the cells, thereby reducing the pathogenesis of atherosclerosis and/or vascular calcification. Other embodiments comprise measuring hedgehog signaling in the cells. The target cells may be in culture or may be cells of a subject, e.g., a human or other animal.

The methods described herein may involve administering to a subject a hedgehog signaling modulating agent selected from a recombinant hedgehog protein and an oxysterol through either systemic or local delivery to the target cells. In certain embodiments, local delivery may include the use of injections and/or implants, such as stents, which are often implanted following balloon angioplasty procedures. Merely by way of example, the compositions described herein may be coated onto an implant device, such as a stent or catheter, for delivery into an atherosclerotic site. Other methods may involve modulating hedgehog signaling in target cells by local manipulation of gene expression through gene therapy approaches.

In certain embodiments, the methods may involve treating vascular or immune cells, comprising contacting vascular or immune cells with an agent selected from a recombinant hedgehog protein and an oxysterol in an amount effective to modulate hedgehog signaling, and measuring hedgehog signaling in the cells. In other embodiments, the methods may comprise increasing, preventing or reducing the activation of hedgehog signaling, and/or the expression of its target genes in atherosclerotic lesions, vascular cells, or immune cells, and/or the amount of hedgehog gene products present therein. Certain methods of modulating hedgehog signaling, as provided herein, may intervene with atherosclerosis and vascular calcification at the systemic level. Certain methods of modulating hedgehog signaling may impact artery wall lipid and lipoprotein metabolism.

In additional embodiments, the methods described herein may further comprise stimulating or increasing the level of anti-inflammatory molecules that prove to be direct or indirect targets of hedgehog signaling in the artery wall, including cells that are either resident in the artery wall or are recruited to the artery wall. Such anti-inflammatory molecules may include, but are not limited to, various anti-oxidants such as catalase and superoxide dismutase, cytokines with anti-inflammatory properties such as interleukin 10, and other factors that are produced in the artery wall or stimulated in peripheral tissues and carried to the artery wall. Such anti-inflammatory molecules may also act on various steps of lipoprotein metabolism in the artery wall or in peripheral tissues and organs, resulting in a more favorable lipid and lipoprotein profile to help prevent or reverse atherosclerosis and/or vascular calcification. An agonist/activator of hedgehog signaling may be purmorphamine. An agonist/activator of hedgehog signaling may be products of hedgehog genes such as Sonic hedgehog, Indian hedgehog, and Desert hedgehog, or biologically active variants or fragments thereof.

Certain aspects may comprise inhibiting oxysterol-induced osteoblastic differentiation of vascular cells, e.g., in atherosclerotic lesions, thereby reducing vascular calcification, e.g., preventing, arresting, or reversing calcification. The vascular cells may be smooth muscle cells, calcifying vascular cells, or myofibroblasts, or bone-derived mesenchymal cells, such as bone marrow stromal cells.

Additional aspects may comprise reducing chronic inflammatory responses that culminate in atherosclerotic lesion formation and vascular calcification. Other aspects may involve reducing the amount of Sonic hedgehog protein in atherosclerotic lesions. In certain embodiments, the methods provided herein may reduce vascular calcification.

Methods for modulating a hedgehog (Hh) pathway mediated response in a cell or tissue may comprise contacting the cell or tissue with an effective amount of an oxysterol as disclosed in co-pending PCT International Application PCT/US2007/005073, entitled “Oxysterol Compounds and the Hedgehog Pathway.” The target cell or target tissue may be in vitro or in vivo. The target cells may be resident cells of the artery wall or those recruited to that site during atherosclerosis, including, but not limited to, human endothelial cells, monocyte/macrophages, T cells, myofibroblasts, calcifying vascular cells, pericytes, vascular smooth muscle cells. The Hh pathway mediated response may be a reduction in atherosclerosis.

Further embodiments include methods for treating a subject with a cardiovascular disease or disorder comprising administering to the subject an effective amount of a pharmaceutical composition comprising a hedgehog signaling modulator, such as a recombinant hedgehog protein and/or an oxysterol, at a therapeutically effective dose in an effective dosage form at a selected interval to reduce atherosclerosis associated with the cardiovascular disease or disorder.

Another aspect of the invention relates to methods for identifying a modulator of a hedgehog pathway-mediated activity, comprising screening candidate agents, such as recombinant hedgehog proteins or oxysterols, for the ability to modulate an activity in one of the hedgehog-related in vitro or in vivo assays discussed herein, such as by measuring immunohistochemical staining of lesions for diagnostic targets of Hh signaling, or by measuring the effects of a candidate agent on lipopolysaccharide (LPS)-induced macrophage activation.

Another aspect of the invention relates to complexes (in vitro or in vivo) comprising an oxysterol of the present invention and any of a variety of intracellular oxysterol binding molecules (e.g., proteins, receptors, etc.), especially those binding molecules found in an atherosclerotic plaque or lesion, examples of which will be evident to a person skilled in the art.

A method according to the invention includes treating a cardiovascular disease, including administering to a subject a therapeutically effective amount of a composition including a modulator of hedgehog activity. The subject can have or be at risk of having the cardiovascular disease. The modulator can be an agonist, activator, antagonist, and/or inhibitor. The modulator can be selected from the group consisting of an inhibitory oxysterol, cyclopamine, cyclopamine-KAAD, Jervine, Tomatidine HCl, and SANT-1. The modulator can be, for example, an oxysterol, purmorphamine, and products of hedgehog genes such as sonic hedgehog, indian hedgehog, and desert hedgehog. The modulator can be, for example, Oxy1, Oxy2, and/or Oxy16. The modulator can be, for example, 3-cholesten-3beta, 5-cholesten-3beta, 20alpha-diol-3-acetate, 4beta-hydroxycholesterol, 20S-hydroxycholesterol, 22S-hydroxycholesterol, 22R-hydroxycholesterol, 24-hydroxycholesterol, 24S,25epoxycholesterol, 26-hydroxycholesterol, and/or pregnanolone. The modulator can be, for example, Oxy3, Oxy4, Oxy7, Oxy8, Oxy9, Oxy10, and/or Oxy11. The modulator can be, for example, Oxy12, Oxy13, Oxy14, Oxy15, Oxy20, Oxy22, Oxy26, Oxy27, Oxy28, Oxy34, Oxy36, Oxy38, Oxy39, Oxy 40, Oxy41, Oxy42, Oxy48, and/or Oxy49. The modulator can be, for example, 4alpha-hydroxycholesterol, 7alpha-hydroxycholesterol, and/or 7-keto-hydroxycholesterol. The modulator can be, for example, 24S-hydroxycholesterol and/or 25-hydroxycholesterol. The modulator can be, for example, Oxy6. The modulator can be, for example, Oxy5 and/or Oxy17. The modulator can be, for example, a combination of any two or more of the above compounds.

A method according to the invention includes reducing the pathogenesis of atherosclerosis, including contacting vascular cells, immune cells, and/or stem cells with a recombinant hedgehog protein, a biologically active fragment or variant of a recombinant hedgehog protein, or an oxysterol in an amount effective to modulate hedgehog signaling in the cells, thereby reducing the pathogenesis of atherosclerosis. The vascular cells can be, for example, mature endothelial cells, progenitor endothelial cells, vascular smooth muscle cells, pericytes, adipocytes, calcifying vascular cells, myofibroblasts, and/or pluripotent mesenchymal cells. The immune cells can be, for example, monocytes, macrophages, T cells, B cells, and/or dendritic cells. The stem cells can be, for example, mesenchymal stem cells, bone marrow stromal cells, and/or hematopoietic stem cells.

### BRIEF DESCRIPTION OF THE DRAWINGS

FIG. 1 shows immunohistochemical analysis of Ptch and Gli1 expression in mouse atherosclerotic lesions and normal aorta. Frozen sections of mouse atherosclerotic lesions were stained with anti-Ptch antibody (A,D), anti-Gli1 antibody (B,E), anti-Shh antibody (F) or secondary antibody alone as negative control (C).

FIG. 2 shows immunohistochemical analysis of Ptch expression in human atherosclerotic lesion. Frozen sections of human atherosclerotic lesions were stained with anti-Ptch antibody (A) or secondary antibody alone as a negative control (B).

FIG. 3 shows immunohistochemical analysis of Ptch expression in human calcified atherosclerotic lesion. Frozen sections of human atherosclerotic lesions obtained from atherectomy were stained with anti Ptch antibody (A) or secondary antibody alone as a negative control (B).

FIG. 4 shows the effect of Shh on Ox PAPC induced IL8 mRNA expression in human aortic endothelial cells at 4 hours (4A) and 16 hours (4B).

FIG. 5 shows the inhibitory effects of Shh protein on the activation of macrophages by lipopolysaccharide (LPS) as measured by tumor necrosis factor-α (TNF-α) expression.

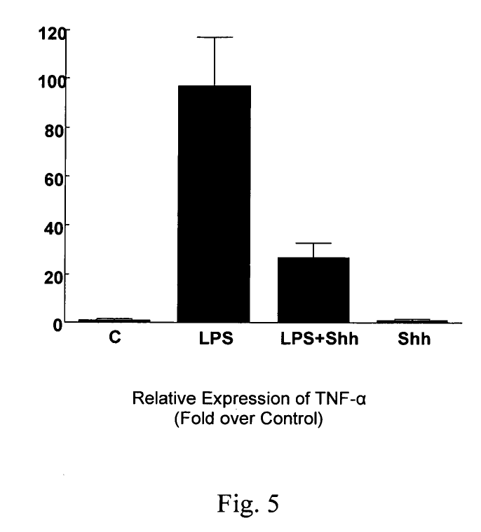

FIG. 6 shows the detailed chemical structure of representative synthetic oxysterols Oxy1 through Oxy 4 and Oxy6 through Oxy11.

FIG. 7 shows the detailed structure of representative oxysterols Oxy12 through Oxy 16.

FIG. 8 shows the detailed structure of representative oxysterols Oxy22, Oxy26, and Oxy27.

FIG. 9 shows the detailed structure of representative oxysterols Oxy28, Oxy39, Oxy40, Oxy41, Oxy 42, Oxy48, and Oxy49.

FIG. 10 shows the detailed structure of representative oxysterols Oxy20, Oxy34, Oxy36, and Oxy38.

FIG. 11 shows the detailed structure of representative oxysterols Oxy5 and Oxy17.

### DETAILED DESCRIPTION

Prior to setting forth the invention, it may be helpful to an understanding thereof to set forth definitions of certain terms that will be used hereinafter.

As used herein, the singular forms “a,” “an” and “the” include plural referents unless the context clearly dictates otherwise. For example, “an” oxysterol, or “a” recombinant hedgehog protein, includes multiple oxysterols or proteins (e.g. 2, 3, 4, 5 or more oxysterols or recombinant proteins), which can be the same or different, and which, for the can be synthetic or naturally occurring.

A “subject,” as used herein, includes any animal that exhibits a symptom that can be treated with an oxysterol of the invention. Suitable subjects (patients) include laboratory animals (such as mouse, rat, rabbit, or guinea pig), farm animals, and domestic animals or pets (such as a cat or dog). Non-human primates and human patients are included. Typical subjects include animals that exhibit aberrant amounts (lower or higher amounts than a “normal” or “healthy” subject) of one or more physiological activities that can be modulated by an oxysterol of the invention (e.g., formation of atherosclerotic plaques). For example, a “subject” may have atherosclerotic lesions and/or vascular calcification at one or more arterial sites. A subject may also be undergoing, or have undergone, treatment for atherosclerotic lesions (e.g., medication and/or surgery) and/or may have certain clinical risk factors (e.g., clinical markers of atherosclerosis, genetic predisposition) that increase their risk of developing atherosclerosis and/or vascular calcification.

The ability of an agent, as provided herein, to “modulate” a response includes the ability to increase or to decrease the level of the response compared to the response elicited in the absence of the agent. The aberrant activities may be regulated by any of a variety of mechanisms, including activation of a hedgehog activity, macrophage activation, etc. The aberrant activities can result in a pathological condition.

As used herein, there are various types of modulators. An “agonist” is a compound that stimulates a physiological response, for example, by binding to a receptor. For example, a hedgehog agonist stimulates the hedgehog pathway. An “activator” is a compound that stimulates a physiological response, for example, by binding to DNA to increase the rate of transcription or binding to an enzyme to increase its activity. An “enhancer” is a compound that increases the effect of another modulator. An “antagonist” reverses or negates the action of an agonist, for example, by binding to a receptor of the agonist and blocking or damping the agonist-mediated response. An “inhibitor” is a compound that decreases a physiological response, for example, by binding to an enzyme and decreasing its activity.

An “effective amount,” as used herein, includes an amount that can bring about a detectable effect. A “therapeutically effective amount,” as used herein, includes an amount that can bring about a detectable therapeutic effect (e.g., the amelioration or reduction of a symptom).

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements.

An “implant,” as used herein, refers to a device or object that is implanted or inserted into a body. Examples of implants include, but are not limited to, angioplasty balloons, stents, drug-eluting stents, vascular catheters, dialysis catheters, vascular grafts, prosthetic heart valves, cardiac pacemakers, implantable cardioverter defibrillators. When used according to exemplary methods provided herein, an implant may contain, or be coated with, an Hh modulating agent, such as a recombinant Hh protein, a biologically active fragment or variant thereof, or an oxysterol.

As used herein, the terms “function” and “functional” and the like refer to a biological, enzymatic, or therapeutic function. The present invention contemplates the use in the methods present application of recombinant hedgehog protein sequences as well as their biologically active fragments. Typically, biologically active fragments of a recombinant hedgehog protein or polypeptide may participate in an interaction, for example, an intra-molecular or an inter-molecular interaction. An inter-molecular interaction can be a specific binding interaction or an enzymatic interaction (e.g., the interaction can be transient and a covalent bond is formed or broken).

“Polypeptide”, “peptide,” “protein” and “recombinant protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers.

The recitation polypeptide “variant” refers to polypeptides that are distinguished from a reference polypeptide by the addition, deletion or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues.

“Treatment” or “treating,” as used herein, includes any desirable effect on the symptoms or pathology of a disease or condition, and may include even minimal reductions in one or more measurable markers of the disease or condition being treated. “Treatment” does not necessarily indicate complete eradication or cure of the disease or condition, or associated symptoms thereof. “Treating atherosclerosis,” as used herein, may include regressing or decreasing the formation or presence of atherosclerotic lesions, and may also include prophylaxis for preventing or reducing the risk of formation of atherosclerotic lesions in a subject. The terms atherosclerotic “plaques” and “lesions” are used interchangeably.

The present invention relates to the surprising discovery that recombinant hedgehog proteins and oxysterols modulate Hedgehog (Hh) signaling activity, and in particular relates to where such Hh signaling activity is associated with cardiovascular diseases. In certain aspects, the cardiovascular diseases include the formation or presence of atherosclerotic lesions or plaques. In certain examples provided herein, oxysterols stimulate the hedgehog (Hh) signaling pathway. In other embodiments, oxysterols and/or recombinant Hh proteins, or biologically active fragments or variants thereof, inhibit or reduce the activation of atherosclerotic lesion-producing macrophages. In various embodiments, the formation or presence of atherosclerotic lesions or plaques is associated with a cardiovascular disease, disorder, or condition, and the methods provided herein relate to treating or reducing the symptoms and/or pathogenesis of such cardiovascular diseases, disorders, or conditions.

Since Hh activity is associated with atherosclerotic lesions, oxysterols or recombinant Hh proteins that can act either in vitro or in vivo (in a subject) to modulate (e.g., cause to increase/stimulate/enhance or cause to decrease/inhibit) this pathway. Naturally occurring molecules as well as synthetic molecules, or combinations thereof, may be used to treat or modulate atherosclerotic conditions mediated by elements of the Hh pathway, and may thus be used to treat or prevent related cardiovascular conditions.

Oxysterols of the invention can be inexpensive to manufacture, can be easily administered (e.g., locally or systemically), and can exhibit great efficacy and potency. Representative oxysterol compounds that may be used according to the embodiments of the present invention are described herein, and are also described in PCT/US2007/005073, which is hereby incorporated by reference in its entirety. Representative hedgehog protein sequences that may be used to isolate biologically active recombinant hedgehog proteins, for use with the methods provided herein, are well-known to, and readily derivable by, a person skilled in the art, and include biologically active Sonic hedgehog, Indian hedgehog, and Desert hedgehog protein sequences.

### Cardiovascular Diseases

Cardiovascular and/or vascular diseases, disorders or conditions, as used herein, relate generally to any condition that affects the heart or blood vessels, or is derived from activity (e.g., cellular activity) associated with the heart or blood vessels, such as immune cell activity. In certain aspects, a cardiovascular or vascular condition could have an effect on other organs or areas of the body, such as the skin, bones, joints, lungs, kidneys, muscle tissue, the gastrointestinal tract, among others known to a person skilled in the art. In certain aspects, the cardiovascular or vascular disease or condition is associated with atherosclerosis and/or the activation of macrophages.

Atherosclerosis is a chronic inflammatory disease that is triggered by the accumulation of specific species of oxidized lipids that are derived from lipoproteins and cellular lipids (Ross, Nature 362:801-809 (1993); Badimon et al., Curr Mol Med 6:439-456 (2006); and Libby, Nature 420:868-874 (2002)). Accumulation of such inflammatory lipids activates the aortic EC to express cytokines and adhesion molecules that specifically recruit and bind monocytes, resulting in their transmigration into the subendothelial space, foam cell development and fatty streak formation. The progression of lesions brings about greater insult to vascular cells, production of inflammatory cytokines, and the differentiation of a population of pluripotent mesenchymal cells (a subpopulation of smooth muscle cells/myofibroblasts) into osteoblastic cells that have the hallmarks of bone osteoblasts and form a mineralized matrix (Shao et al., Arterioscler Thromb Vasc Biol 26:1423-1430 (2006); and Demer et Curr Opin Nephrol Hyperten 11:437-443 (2002)).

Although these calcifying vascular cells (CVC) are similar in many respects to bone osteoblasts, they also differ from their bone counterparts by having reciprocal responses to certain inflammatory molecules, including minimally oxidized-low density lipoprotein (MM-LDL) and the inflammatory cytokines TNF-α and IL-6 (Parhami et al., Arterioscler Thromb Vasc Biol 17:680-687 (1997); and Tintut et al., Circulation 102:2636-2642 (2000)). For example, these factors induce osteoblastic differentiation of CVC, whereas they inhibit such differentiation of bone marrow derived osteoprogenitor marrow stromal cells (MSC). As illustrated by the Examples provided herein, activation of hedgehog signaling is detectable specifically in atherosclerotic lesions and/or sites of vascular calcification (i.e., it is not significantly detectable in normal vascular tissues), and thus represents a target for therapeutic intervention.

The formation or presence of atherosclerotic lesions and/or vascular calcification, as described above, is associated with a variety of cardiovascular diseases, disorders, or conditions. Such “cardiovascular diseases” and/or “vascular diseases,” as used herein, may encompass any cardiovascular or vascular disease, disorder or condition associated with, related to, or caused by the formation or presence of atherosclerotic plaques and/or vascular calcification, and/or may encompass any vascular disease, disorder, or condition relating to the activation of macrophages. Examples of cardiovascular diseases and vascular diseases include, but are not limited to, aneurysms, angina, arrhythmia, atherosclerosis, cardiomyopathy (e.g., alcoholic, ischemic, valvular, inflammatory), stroke, cerebrovascular disease, chronic inflammatory diseases (e.g., rheumatoid arthritis, osteoarthritis, inflammatory lung disease, these inflammatory bowel disease, psoriasis), congenital heart disease, congestive heart failure, coronary artery disease, myocarditis, valve disease, dilated cardiomyopathy, diastolic dysfunction, endocarditis, hypertension, hypertrophic cardiomyopathy, inflammatory heart disease, ischemic heart disease, macrophage activation syndrome, mitral valve prolapse, myocarditis, myocardial infarction (heart attack), venous thromboembolism, peripheral artery occlusive disease, stenosis, and restenosis.

### Hedgehog Signaling

In mammals, three members of the Hh family of proteins have been identified, including Sonic hedgehog (Shh), Indian hedgehog (Ihh) and Desert hedgehog (Dhh, mainly present in neural tissues). In addition to its role in embryonic development, Hh signaling plays a crucial role in postnatal development and maintenance of tissue/organ integrity and function.

Hh signaling involves a very complex network of factors that includes plasma membrane proteins, kinases, phosphatases, and factors that facilitate the shuttling and distribution of hedgehog molecules. Production of Hh proteins from a subset of producing/signaling cells involves synthesis, auto-processing and lipid modification. In the absence of Hh proteins, Patched (Ptch), present on the plasma membrane of the responding cells, keeps Hh signaling in a silent mode by inhibiting the activity of another plasma membrane-associated signal transducer molecule, Smoothened (Smo). In the presence of Hh, the inhibition of Smo by Ptch is alleviated and Smo transduces the signal that regulates the transcription of Hh target genes. This transcriptional regulation in part involves the Ci/Gli transcription factors that enter the nucleus from the cytoplasm after a very intricate interaction between the members of a complex of accessory molecules that regulate the localization of Gli. Genes that are targeted by Hh signaling include Gli1, Ptch, bone morphogenetic protein 2 (BMP2), Wnt and homeobox genes.

Hedgehog signaling plays a role in osteoblastic differentiation of vascular cells, which represents an important step in vascular calcification. In addition, the Examples provided herein demonstrate that the hedgehog signaling pathway is highly activated in advanced atherosclerotic lesions from mice and humans, and further provide a person skilled in the art with methods for measuring hedgehog signaling in the same. For example, staining of atherosclerotic tissues for Ptch and Gli1, diagnostic markers for Hh signaling, showed expression of these markers around both areas of calcification and areas represented by osteoid-like matrices along the internal elastic lamina. These areas well known for being associated with the initiation and progression of vascular calcification. In contrast, immunohistochemical examination of normal arterial tissue did not show staining for Ptch and Gli. These results demonstrate a relationship between vascular calcification (i.e., atherosclerosis) and the activation of hedgehog signaling pathways, and suggest that hedgehog signaling plays an important role in the molecular regulation of vascular calcification.

The Applicants have further demonstrated that the hedgehog signaling pathway is robustly activated by oxysterols, which have osteoinductive properties when applied to pluripotent mesenchymal cells derived from the bone marrow. These mesenchymal cells, which serve as osteoprogenitors during skeletal development and bone remodeling, are induced to undergo osteoblastic differentiation when treated with osteoinductive oxysterols. Oxysterol-induced osteoblastic differentiation of cells is dependent on the activation of hedgehog signaling pathway, and is completely inhibited by the hedgehog pathway inhibitor, cyclopamine.

Hedgehog signaling also plays a role in the regulation of inflammation. For example, misregulated hedgehog signaling appears to be involved in chronic inflammatory processes in the gastrointestinal tract and in the lung, involving epithelial cells, monocytes, and T lymphocytes. In addition, hedgehog signaling modulates the activation of and cytokine production by T cells. Furthermore, endothelial cells represent direct and/or indirect targets of hedgehog signaling not only during embryonic vasculogenesis, but during post-embryonic angiogenesis as well, which occurs in a variety of settings, such as in ischemic tissues. In particular, the Examples provided herein demonstrate that Hh signaling modulates inflammatory cytokine production in aortic endothelial cells (EC), and targets the expression of downstream molecules in both human aortic smooth muscle cells (HASMC) and mouse aortic smooth muscle cells (MASMC). Accordingly, hedgehog signaling may play an important role in regulating the chronic inflammatory responses that culminate in atherosclerotic lesion formation, such that modulating hedgehog signaling can modulate the inflammatory response.

Further related to inflammatory responses, Hh signaling plays a role in macrophage activation. For example, Hh signaling via Shh protein inhibits the lipopolysaccharide (LPS)-induced activation of macrophages, as measured by TNF-α expression. Macrophage activation represents a hallmark of atherosclerosis, such that modulating Hh signaling-related macrophage activation may modulate the formation of atherosclerotic lesions.

Accordingly, activation of Hh signaling in the cells associated with vascular tissues, including vascular mesenchymal cells (i.e., SMC/CVC), endothelial cells and/or macrophages, whether by oxysterols or by recombinant hedgehog proteins, modulates the inflammatory responses of these vascular cells to other atherogenic and/or osteoinductive factors, such as oxidized lipids and cytokines.

The molecular mechanism(s) that mediate hedgehog signaling in the vessel wall represent therapeutic targets related to the methods provided herein. Accordingly, certain methods described herein encompass targeting vascular cells to intervene in the pathway by which Hh signaling modulates the responses of these cells to inflammatory molecules that participate in the formation of atherosclerotic lesions and vascular calcification.

### Active Agents

a. Hedgehog Proteins and Fragments

Hedgehog (hh) proteins represent a family of secreted signal proteins responsible for the formation of numerous structures in embryogenesis (see, e.g., Smith, Cell 76 (1994) 193-196; Perrimon, Cell 80 (1995) 517-520; Chiang et al., Nature 83 (1996) 407; Bitgood et al., Curr. Biol. 6 (1996) 298-304; Vortkamp et al., Science 273 (1996) 613; and Lai et al., Development 121 (1995) 2349). During biosynthesis, signal sequence cleavage and autocatalytic cleavage form a 20 kDa N-terminal domain and a 25 kDa C-terminal domain. In its natural form, the N-terminal domain is modified with cholesterol or palmitoyl (see, e.g., Porter et al., Science 274 (1996) 255-259; Pepinski et al., J. Biol. Chem. 273 (1998) 14037-14045). In higher life-forms the Hh family is composed of at least three members, including Sonic, Indian and Desert hedgehog (Shh, Ihh, Dhh; M. Fietz et al., Development (Suppl.) (1994) 43-51).

The present invention relates in part to the unexpected discovery that recombinant hedgehog proteins, or biologically active fragments or variants thereof, are capable of inhibiting lipopolysaccharide (LPS)-induced macrophage activation by activating the hedgehog signaling pathway, and thereby inhibiting the formation of atherosclerotic plaques, since macrophage activation is a hallmark of atherosclerosis. Representative examples of recombinant hedgehog proteins that may be used according to the methods provided herein include, but are not limited to, Sonic hedgehog (Shh), Indian hedgehog (Ihh), and Desert hedgehog (Dhh), in addition to combinations thereof.

Recombinant proteins may be routinely produced or manufactured according to well-known molecular biological techniques in the art. For example, methods known to those skilled in the art may be employed to construct expression vectors containing sequences encoding a recombinant hedgehog protein of interest, and may be further employed to express and purify the recombinant hedgehog proteins encoded therein. Such methods include in vitro recombinant DNA techniques, synthetic techniques, in vivo genetic recombination, and protein expression and purification techniques, which are described, for example, in Sambrook and Russell (2001) Molecular Cloning, A Laboratory Manual, 3^{rd} edition (Cold Spring Harbor Press, Plainview, N.Y.).

The present methods contemplate the use of full-length recombinant hedgehog proteins, in addition to biologically active fragments or variants thereof. Biologically active fragments of a full-length hedgehog protein include polypeptides comprising amino acid sequences sufficiently similar to, or derived from, the amino acid sequences of a (putative) full-length hedgehog protein. Typically, biologically active fragments comprise a domain or motif with at least one activity of a full-length hedgehog polypeptide and may include one or more (and in some cases all) of the various active domains, and include fragments having fragments having a hedgehog modulating activity, such as the ability to inhibit LPS-induced macrophage activation.

A biologically active fragment of a full-length hedgehog protein or polypeptide can be a polypeptide which is, for example, 10, 11, 12, 13, 14, 15, 16, 17, 18, 19, 20, 21, 22, 23, 24, 25, 26, 27, 28, 29, 30, 40, 50, 60, 70, 80, 90, 100, 120, 150, or more contiguous amino acids of the known hedgehog sequences, whether from Sonic hedgehog, Indian hedgehog, or Desert hedgehog polypeptide sequences. Suitably, the biologically-active fragment has no less than about 1%, 10%, 25% 50% of an activity of the full-length polypeptide from which it is derived.

b. Oxysterols

Oxysterols comprise a large family of oxygenated derivatives of cholesterol that are present in the circulation of animal tissues (Edwards et al., Annu Rev Biochem 68:157-185 (1999); and Bjorkhem et al., Curr Opion Lipidol 13:247-253 (2002)). Oxysterols may be formed by auto-oxidation, as a secondary byproduct of lipid peroxidation, or by the action of specific monooxygenases, most of which are members of the cytochrome P450 family of enzymes. Cytochrome P450 enzymes are also involved in the further oxidation and metabolism of oxysterols into active or inactive metabolites, which eventually leads to their removal from the system.

Oxysterols that have been identified in human plasma to date include 7α-hydroxycholesterol, 25-hydroxycholesterol, and 4α- and 4β-hydroxycholesterol, which are present at concentrations ranging from 5-500 ng/ml. These oxysterols have a variety of half-lives in circulation ranging from 0.5-60 hours, and their levels can be altered by aging, drug interventions, and disease processes. Examples of these enzymes include cholesterol 7α-hydroxylase (CYP7A1) that forms 7α-hydroxycholesterol, cholesterol 25-hydroxylase that forms 25-hydroxycholesterol, cholesterol 24S-hydroxylase (CYP46) that forms 24S′-hydroxycholesterol, and others. In addition, oxysterols may be derived from the diet.

A role for specific oxysterols has been implicated in various physiologic processes, including cellular differentiation, inflammation, apoptosis, and steroid production. The main identified receptor(s) for certain oxysterols that upon activation results in regulation of transcription of target genes is the liver X receptor (LXRα and LXRβ) (Zelcer et al., J Clin Invest 116:607-614 (2006)). Many of the effects of oxysterols are not explained by LXR activation, including their osteoinductive and anti-adipogenic effects on mesenchymal cells (Kha et al., J Bone Miner Res 19:830-840 (2004)).

The downstream effects of oxysterols are mediated through the activation of Hh signaling in pluripotent mesenchymal cells. Accordingly, as noted herein, it is believed that these oxysterols also activate Hh signaling in vascular mesenchymal cells (i.e., SMC/CVC) and endothelial cells, and that this activation of Hh signaling by oxysterols or Shh modulates the inflammatory responses of these cells to other atherogenic and/or osteoinductive factors. Not wishing to be bound by any one theory, Hh signaling may regulate osteoblastic differentiation of vascular cells, such that osteoinductive oxysterols may induce osteoblastic differentiation of vascular cells through an Hh signaling-dependent mechanism. In addition, oxysterols may regulate their pro-inflammatory responses in aortic endothelial cells via activation of Hh signaling.

Examples of oxysterols that may by used according to the methods provided herein include both naturally occurring and synthetic oxysterols. Naturally occurring oxysterols may be “isolated” before use according to the methods provided herein. By “isolated” is meant removed from its original environment (e.g., the natural environment if it is naturally occurring), and/or separated from at least one other component with which it is naturally associated. For example, a naturally-occurring oxysterol present in its natural living host is not isolated, but the same oxysterol, separated from some or all of the coexisting materials in the natural system, is isolated. Such an oxysterol can be part of a composition (e.g., a pharmaceutical composition), and still be isolated in that such composition is not part of its natural environment. Also, an intermediate product in the synthesis of another oxysterol, wherein the intermediate product is not purified or separated from other components in the reaction pathway, is not isolated.

Examples of naturally occurring oxysterols include, but are not limited to, 22(S)-hydroxycholesterol (sometimes referred to herein as “22S”); 22(R)-hydroxycholesterol (sometimes referred to herein as “22R”); 20(S)-hydroxycholesterol (also known as 20-alpha hydroxycholesterol, and sometimes referred to herein as “20S”); 5-cholesten-3beta, 20alpha-diol 3-acetate; 24-hydroxycholesterol; 24(S), 25-epoxycholesterol; pregnanolone, 26-hydroxycholesterol; 4beta-hydroxycholesterol; can also be used.

Examples of synthetic oxysterols include, but are not limited to, those described in co-pending International Application PCT/US2007/005073, such as oxysterols represented by Formula I below:

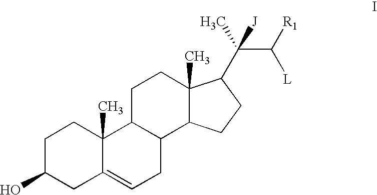

wherein J is H or OH,

wherein L is H or OH,

wherein at least one of J and L is H,

wherein at least one of J and L is OH, and

wherein R1 is selected from the group consisting of alkane of from 1 to 6 carbons, alkene of from 2 to 6 carbons, and phenyl optionally substituted with methyl,

optionally provided that R1 is not 3-methylbutyl,

optionally provided that when J is OH, R1 is not 3-methyl-2-butenyl, and

optionally provided that when L is OH, R1 is not n-propyl.

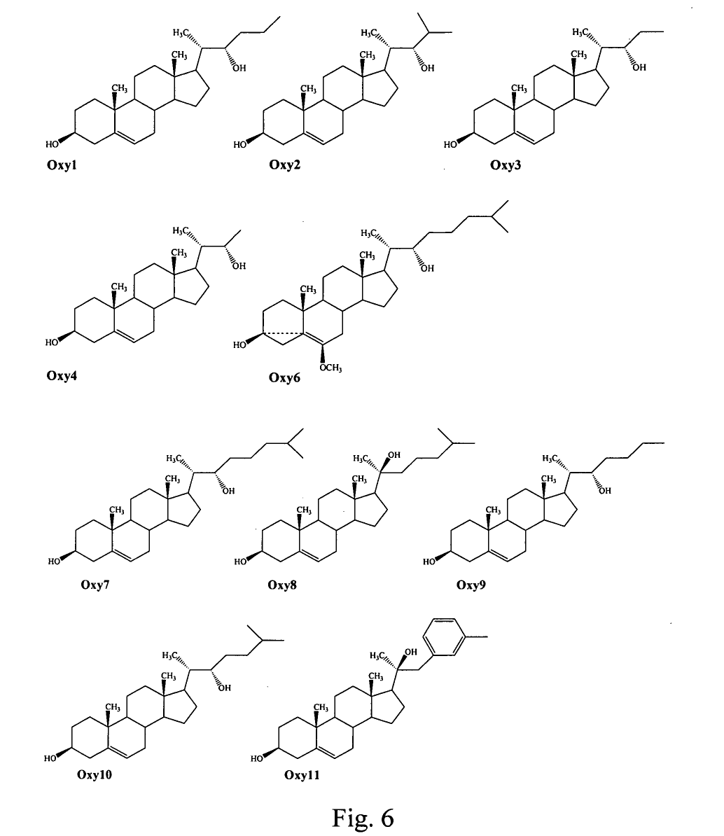

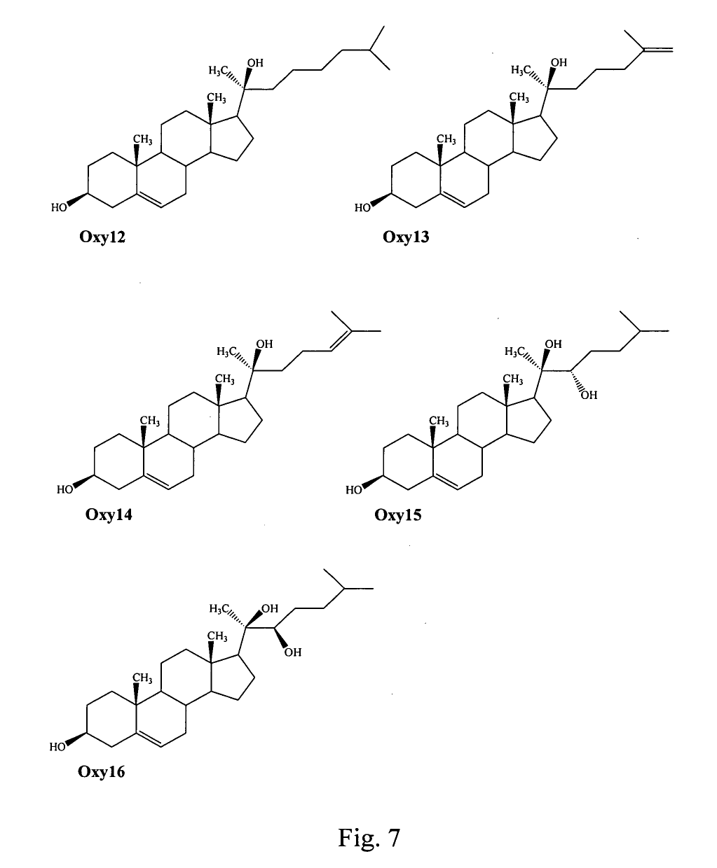

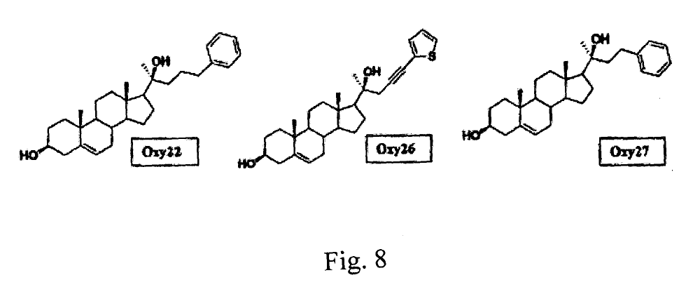

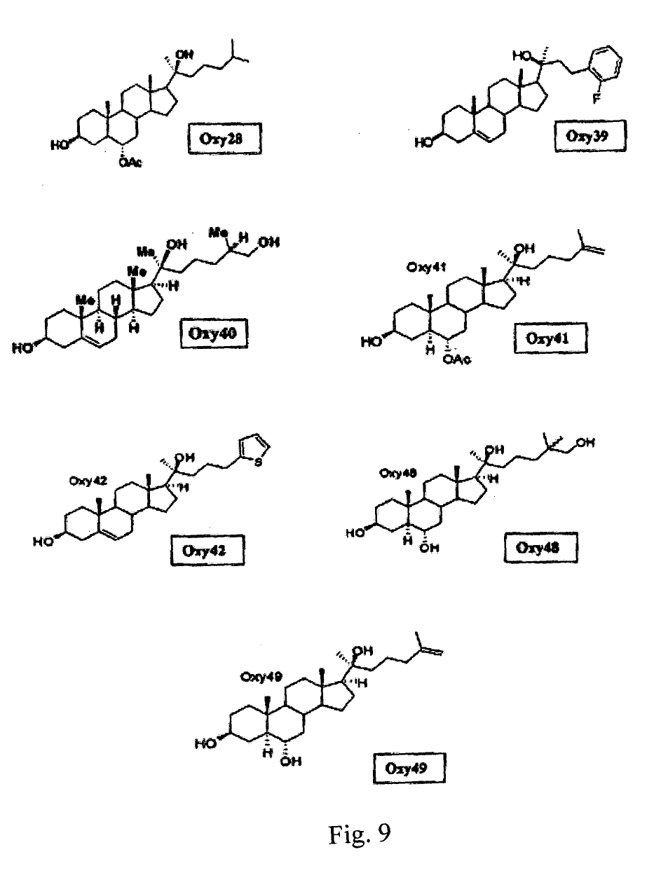

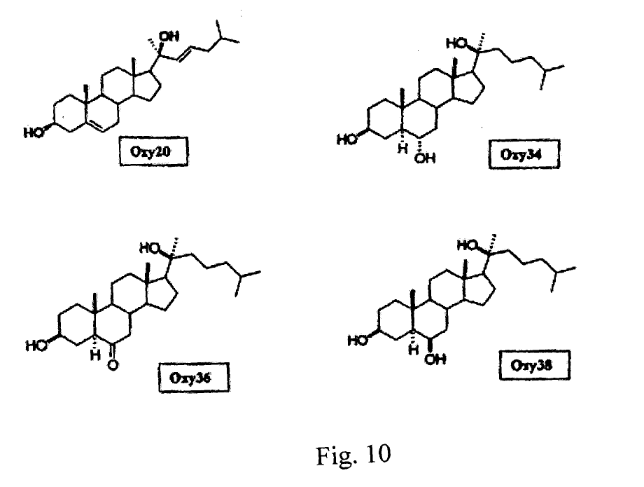

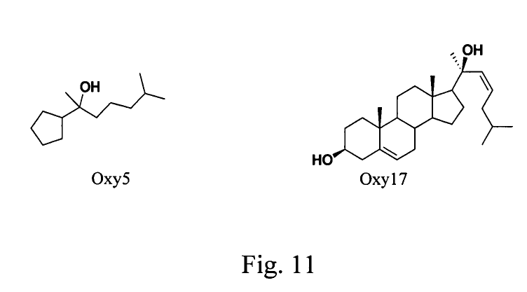

Examples of synthetic oxysterols that may be used as provided herein include, for example, Oxy1, Oxy2, Oxy3, Oxy4, Oxy6, Oxy7, Oxy8, Oxy9, Oxy10, Oxy11, Oxy12, Oxy13, Oxy14, Oxy15, and Oxy16, as represented in FIGS. 6 and 7. Examples of synthetic oxysterols that may be used as provided herein include, for example, Oxy 5, Oxy 17, Oxy20, Oxy22, Oxy26, Oxy27, Oxy28, Oxy34, Oxy36, Oxy38, Oxy39, Oxy40, Oxy41, Oxy42, Oxy48, and Oxy49, as represented in FIGS. 8, 9, 10, and 11.

Combinations of two or more oxysterols, with one another and/or with other oxysterols, including naturally occurring oxysterols, may also be used in methods of the invention.

Oxysterols and other compounds can function as modulators of hedgehog activity. Oxysterols and other compounds can function as agonists, activators, and/or enhancers of hedgehog activity. Oxysterols and other compounds can function as antagonists and/or inhibitors of hedgehog activity.

For example, a compound such as purmorphamine can function as an agonist and/or activator of hedgehog activity. For example, products of hedgehog genes such as sonic hedgehog, indian hedgehog, and desert hedgehog can function as agonists and/or activators of hedgehog activity. For example, compounds such as 3-cholesten-3beta, 5-cholesten-3beta, 20alpha-diol-3-acetate, 4beta-hydroxycholesterol, 20S-hydroxycholesterol, 22S-hydroxycholesterol, 22R-hydroxycholesterol, 24-hydroxycholesterol, 24S,25epoxycholesterol, 26-hydroxycholesterol, and pregnanolone can function as agonists and/or activators of hedgehog activity. For example, synthetic oxysterols such as Oxy3, Oxy4, Oxy7, Oxy8, Oxy9, Oxy10, and Oxy11 can function as agonists and/or activators of hedgehog activity. For example, synthetic oxysterols such as Oxy12, Oxy13, Oxy14, Oxy15, Oxy20, Oxy22, Oxy26, Oxy27, Oxy28, Oxy34, Oxy36, Oxy38, Oxy39, Oxy 40, Oxy41, Oxy42, Oxy48, and Oxy49 can function as agonists and/or activators of hedgehog activity.

For example, compounds such as cyclopamine, cyclopamine-KAAD, Jervine, Tomatidine HCl, and SANT-1 can function as antagonists and/or inhibitors of hedgehog activity. For example, synthetic oxysterols such as Oxy1, Oxy2, and Oxy16 can function as antagonists and/or inhibitors of hedgehog activity.

For example, compounds such as 4alpha-hydroxycholesterol, 7alpha-hydroxycholesterol, and 7-keto-hydroxycholesterol can function as modulators of hedgehog activity. For example, compounds such as 24S-hydroxycholesterol and 25-hydroxycholesterol can function as modulators of hedgehog activity. For example, synthetic compounds such as Oxy5, Oxy6, and Oxy17 can function as modulators of hedgehog activity.

The Examples herein illustrate some of the activities that are exhibited by oxysterols of the invention. Synthetic and naturally occurring oxysterols, as described herein (e.g., 22(S)-hydroxycholesterol; 22(R)-hydroxycholesterol; 20(S)-hydroxycholesterol; 5-cholesten-3beta, 20alpha-diol 3-acetate; 24-hydroxycholesterol; 24(S), 25-epoxycholesterol; pregnenolone, 26-hydroxycholesterol; 4beta-hydroxycholesterol; and Oxy1 through Oxy16), individually or in combination, can exhibit osteogenic and anti-adipogenic properties, in addition to anti-atherosclerotic properties. Also see, e.g., the compounds in the commonly owned and published PCT international applications WO2004/019884, WO2005/020928, WO2006/110490, WO2007/028101, WO2007/098281, and WO2008/011071, in the international application PCT/US2007/025833, and in the U.S. provisional applications 60/907,001 and 60/996,729, all of which are incorporated herein by reference in their entirety.

c. Hedgehog Encoding Polynucleotides and Gene Therapy

As noted above, agents that are capable of modulating Hh signaling, such as recombinant Hh proteins or biologically active fragments or variants thereof, may be delivered to cells as part of gene delivery vehicles. This may be accomplished by delivery of DNA or cDNA capable of in vivo transcription of the Hh-signaling modulating agent, such as a polynucleotide encoding a recombinant Hh protein, or biologically active fragment or variant thereof. More specifically, in order to produce Hh modulating agents in vivo, a nucleic acid sequence coding for the agent may be placed under the control of a eukaryotic promoter (e.g., a pol III promoter, CMV or SV40 promoter). Where it is desired to more specifically control transcription, the agent may be placed under the control of a tissue or cell specific promoter (e.g., to target cells in the liver), or an inducible promoter, such as metallothionein.

Many techniques for introduction of nucleic acids into cells are known. Such methods include retroviral vectors and subsequent retrovirus infection, adenoviral or adeno-associated viral vectors and subsequent infection, and complexes of nucleic acid with a condensing agent (e.g., poly-lysine). These complexes or viral vectors may be targeted to particular cell types by way of a ligand incorporated into the vehicle. Many ligands specific for cells associated with the vascular wall, such as immune cells and vascular endothelial cells, and other cells are well known in the art.

A wide variety of vectors may be utilized within the context of the present invention, including for example, plasmids, viruses, retrotransposons and cosmids. Representative examples include adenoviral vectors (e.g., WO 94/26914, WO 93/9191; Yei et al., Gene Therapy 1:192-200, 1994; Kolls et al., PNAS 91(1):215-219, 1994; Kass-Eisler et al., PNAS 90(24):11498-502, 1993; Guzman et al., Circulation 88(6):2838-48, 1993; Guzman et al., Cir. Res. 73(6):1202-1207, 1993; Zabner et al., Cell 75(2):207-216, 1993; Li et al., Hum Gene Ther. 4(4):403-409, 1993; Caillaud et al., Eur. J. Neurosci. 5(10):1287-1291, 1993), adeno-associated type 1 (“AAV-1”) or adeno-associated type 2 (“AAV-2”) vectors (see WO 95/13365; Flotte et al., PNAS 90(22):10613-10617, 1993), hepatitis delta vectors, live, attenuated delta viruses and herpes viral vectors (e.g., U.S. Pat. No. 5,288,641), as well as vectors which are disclosed within U.S. Pat. No. 5,166,320. Other representative vectors include retroviral vectors (e.g., EP 0 415 731; WO 90/07936; WO 91/02805; WO 94/03622; WO 93/25698; WO 93/25234; U.S. Pat. No. 5,219,740; WO 93/11230; WO 93/10218).

Within certain aspects of the invention, nucleic acid molecules that encode the agents for modulating Hh signaling may be introduced into a host cell utilizing a vehicle, or by various physical methods. For example, polynucleotides encoding a Hh-signaling modulating agent may be coated onto a medical device (e.g., angioplasty balloon, stent, etc), such as by using a hydrogel or other polymer. Medical device coating techniques and compositions for delivering polynucleotides to cells in vivo are well known in the art (see, e.g., Riessen et al., Hum Gene Ther 4:749-58 (1993)).

Additional examples of such methods include transformation using calcium phosphate precipitation (Dubensky et al., PNAS 81:7529-7533, 1984), direct microinjection of such nucleic acid molecules into intact target cells (Acsadi et al., Nature 352:815-818, 1991), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al., PNAS 89:6094, 1990), lipofection (Feigner et al., Proc. Nall. Acad. Sci. USA 84:7413-7417, 1989), microprojectile bombardment (Williams et al., PNAS 88:2726-2730, 1991), polycation compounds such as polylysine, receptor specific ligands, liposomes entrapping the nucleic acid molecules, spheroplast fusion whereby E. coli containing the nucleic acid molecules are stripped of their outer cell walls and fused to animal cells using polyethylene glycol, viral transduction, (Cline et al., Pharmac. Ther. 29:69, 1985; and Friedmann et al., Science 244:1275, 1989), and DNA ligand (Wu et al, J. of Biol. Chem. 264:16985-16987, 1989), as well as psoralen inactivated viruses such as Sendai or Adenovirus. In one embodiment, the agent for modulating Hh-signaling is introduced into the host cell using a liposome.

### Methods of Treatment

As noted above, the methods provided herein relate generally to the use of hedgehog signaling modulators, such as recombinant hedgehog proteins, biologically active fragments thereof, and/or oxysterols, for treating cardiovascular diseases and/or vascular diseases. Accordingly, embodiments of the present invention encompass the use of such agents, alone or in combination, to modulate hedgehog signaling in target cells involved in the pathogenesis of cardiovascular or vascular diseases. In certain aspects, the cardiovascular diseases and/or target cells are associated with atherosclerotic lesions and/or vascular calcification. In certain aspects, the cardiovascular or vascular disease and/or target cells are associated with the activation of macrophages. The methods provided herein also relate to treating atherosclerosis and/or vascular calcification, such as by regressing or decreasing the formation of arterial atherosclerotic lesions, by administering a hedgehog signaling modulator as described above.

In certain aspects, modulators of hedgehog signaling may be administered systemically, to intervene more generally with atherosclerosis and vascular calcification at the systemic level. Merely by way of example, such systemic administrations may provide a prophylactic effect, by preventing or reducing the formation of atherosclerotic plaques and/or vascular calcification in relatively susceptible or at risk individuals.

In other aspects, the hedgehog signaling modulators provided herein may be administered locally, targeting one or more particularly problematic arterial wall sites, thereby impacting arterial wall lipid and lipoprotein metabolism directly. Merely by way of example, local targeting may be useful in either eliminating or reducing the size of previously formed plaques, and may also be performed in conjunction with other therapeutic modalities, such as angioplasty procedures or other forms of medicative or surgical invention. The present methods also contemplate dual administration techniques, wherein the agents described herein are administered both systemically (e.g., to prevent plaque formation) and locally (e.g., to eliminate plaques or reduce plaque size) in the same subject, to not only treat pre-existing atherosclerotic plaques, but to help prevent or reduce the formation of new plaques.

Such targeting can be achieved through systemic or local delivery of drugs, including the use of medical implants, or through local manipulation of gene expression through gene therapy approaches. Targeting cells involved in the pathogenesis of atherosclerosis and vascular calcification, e.g., cells of the arterial wall, or other vascular or immune cells involved in the pathogenesis of atherosclerosis, is indented to treat atherosclerosis and/or its cellular processes (e.g., to prevent, arrest or reverse the disease process). Particular treatment modalities (i.e., administration routes, dosages, compositions) are known to a person skilled in the clinical arts and described elsewhere herein. Therapeutic modulation of hedgehog signaling in the context of cardiovascular or vascular diseases, such as atherosclerosis, has not been used previously, and represents a novel approach for drug discovery to target vascular cells and limit inflammation in the arterial wall.

In addition, the methods of treatment provided herein may be utilized either alone or in combination with other treatments for cardiovascular diseases and/or atherosclerosis, such as lifestyle monitoring, medications, and/or surgical interventions. For example, the presently available treatments for atherosclerosis, and related cardiovascular conditions, rely heavily on the reduction of risk factors, of which lifestyle factors are of great importance (e.g., smoking cessation, attention to diet, regular exercise and maintenance of appropriate body weight). Medications include the use of anti-hypertensives, which do not solve the underlying problem, but merely control the primary side effects of atherosclerosis, and/or the use of cholesterol reducing drugs. Merely by way of example, if appropriate, the methods provided herein could be used in conjunction with anti-hypertensives to control both the primary side effects (i.e., high blood pressure), and to deal with the underlying atherosclerotic plaques and/or vascular calcification. Surgical interventions include, for example, balloon angioplasty, endarctectomy (i.e., surgical removal of fatty deposits from the arterial walls), and bypass surgery. The presently claimed methods may be employed, for example, during or following balloon angioplasty or other surgical procedures.

Atherosclerosis or vascular calcification, which are often characterized by areas of severe narrowing, or stenosis, may be routinely diagnosed by a person skilled in the art. For example, a clinical or other skilled artisan may employ either angiography or “stress testing,” which have long been the focus of human diagnostic techniques for cardiovascular diseases. Other diagnostic methods include, for example, anatomic detection methods and physiologic measurement methods. Examples of anatomic diagnostic methods include coronary calcium scoring by CT, carotid intimal media thickness (IMT) measurement by ultrasound, and intravascular ultrasound (IVUS). Examples of physiologic diagnostic methods include lipoprotein subclass analysis, glycoslated hemoglobin (HbA1c) analysis, C-reactive protein (hs-CRP) measurements, and homocysteine measurements.

Using such routine techniques, in addition to others known in the art, a person skilled in the art can not only readily determine if a subject has a cardiovascular disease, but can readily determine if the cardiovascular disease is associated with atherosclerosis and/or vascular calcification. Similarly, a person skilled in the art can readily determine whether a subject has developed, or is at risk for developing, atherosclerosis and/or vascular calcification, even if that subject is not presently suffering from a particular cardiovascular disease, such as those described herein. Accordingly, a person skilled in the arts can readily identify candidate subjects for treatment according to the methods provided herein. Moreover, a person skilled in the art can readily monitor the clinical effectivity of the treatments described herein using routine clinical markers for atherosclerotic conditions, including, but not limited to, serum test indicators of c-reactive protein, cholesterol, triglycerides, homocysteine, among others.

### Pharmaceutical Compositions and Devices/Implants

The hedgehog modulators discussed herein can be formulated into various compositions, including pharmaceutical compositions, for use in therapeutic treatment methods. Pharmaceutical compositions can be assembled as a kit. Generally, a pharmaceutical composition comprises an effective amount of a recombinant hedgehog protein and/or an oxysterol, or combination of another agent that may be used in conjunction with a hedgehog modulator as provided herein. An “effective amount” or “therapeutically effective amount,” as used herein, is an amount that is sufficient to effect at least a detectable therapeutic response in the individual over a reasonable time frame. For example, an “effective amount” can ameliorate, at least to a detectable degree, the symptoms of a hedgehog-mediated condition, such as a cardiovascular disease associated with atherosclerosis.

A composition can comprise a carrier, such as a pharmaceutically acceptable carrier. “Pharmaceutically acceptable,” as used herein, refers to a material that is not biologically or otherwise undesirable, i.e., the material may be administered to a subject without causing any significant undesirable biological effects or interacting in a seriously deleterious manner with any of the other components of the pharmaceutical composition in which it is contained. A carrier is normally selected to minimize any degradation of the active ingredient and to minimize any adverse side effects in the subject, according to the understanding of a person skilled in the art. For a discussion of pharmaceutically acceptable carriers and other components of pharmaceutical compositions, see, e.g., Remington's Pharmaceutical Sciences, 18th ed., Mack Publishing Company, 1990, which is incorporated by reference in its entirety.

In one embodiment, the pharmaceutical composition includes at least two (i.e., one or more) oxysterols, including synthetic oxysterols, such as Oxy1, Oxy 2, Oxy 3, Oxy 4, Oxy5, Oxy, Oxy 7, Oxy8, Oxy 9, Oxy10, Oxy 11, Oxy 12, Oxy 13, Oxy 14, Oxy 15, and Oxy16, which are described in PCT/US2007/005073 and known to a person skilled in the art. The pharmaceutical composition may further comprise at least one of 20(S)-hydroxycholesterol, 22(S)-hydroxycholesterol, or 22(R)-hydroxycholesterol, or any other naturally occurring oxysterols. A pharmaceutical composition may also comprise, separately or additionally, one or more recombinant hedgehog proteins, such as Sonic hedgehog, Indian hedgehog, or Desert hedgehog proteins, or biologically active fragments or variants thereof.

A pharmaceutical composition or kit may contain other pharmaceuticals or agents in addition to the oxysterols of the invention. The other agent(s) can be administered at any suitable time during the treatment of the patient, either concurrently or sequentially. One skilled in the art will appreciate that the particular formulation will depend, in part, upon the particular agent that is employed, and the chosen route of administration. Accordingly, there is a wide variety of suitable formulations of compositions of the present invention.

Additional pharmaceuticals or agents may include, for example, parathyroid hormone, sodium fluoride, insulin-like growth factor I (ILGF-I), insulin-like growth factor II (ILGF-II), transforming growth factor beta (TGF-β), a cytochrome P450 inhibitor, a phospholipase activator, arachadonic acid, a COX enzyme activator, an osteogenic prostanoid, an ERK activator, BMP 2, 4, 7 and 14.

Another aspect of the invention is a kit for performing any of the methods discussed herein, comprising one or more recombinant hedgehog proteins or oxysterols of the invention, individually or in combination with one another, or in combination with naturally occurring oxysterols and/or with agents noted herein, optionally packaged in one or more containers. When the kit is for treating a subject, the recombinant hedgehog proteins and/or oxysterol(s) may be in the form of a pharmaceutically acceptable composition. Another aspect of the invention is a method for modulating a hedgehog (Hh) pathway mediated response in a cell or tissue, comprising contacting the cell or tissue with an effective amount of an oxysterol or a pharmaceutical composition of the invention. The cell or tissue may be in vitro or in a subject (in vivo). In the latter case, the subject can be one who would benefit, e.g., from a reduction in atherosclerotic lesions and/or vascular calcification.

Formulations suitable for oral administration can consist of liquid solutions, such as an effective amount of an oxysterol dissolved in diluents, such as water, saline, or fruit juice; capsules, sachets or tablets, each containing a predetermined amount of the active ingredient, as solid, granules or freeze-dried cells; solutions or suspensions in an aqueous liquid; and oil-in-water emulsions or water-in-oil emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers. Suitable formulations for oral delivery can also be incorporated into synthetic and natural polymeric microspheres, or other means to protect the agents of the present invention from degradation within the gastrointestinal tract.

Formulations suitable for parenteral administration (e.g., intravenous) include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (i.e., lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The recombinant hedgehog proteins and/or oxysterols of the present disclosure, alone or in combination with other therapeutic agents, can be made into aerosol formulations to be administered via inhalation. These aerosol formulations can be placed into pressurized acceptable propellants, such as dichlorodifluoromethane, propane, nitrogen, and the like.

Recombinant hedgehog proteins and oxysterols, alone or in combination with other therapeutic agents, can also be made into suitable formulations for transdermal application and absorption. Transdermal electroporation or iontophoresis also can be used to promote and/or control the systemic delivery of the agents and/or pharmaceutical compositions of the present invention through the skin (see, e.g., Theiss et al. (1991), Meth. Find. Exp. Clin. Pharmacol. 13: 353-359).

Suitable formulations for topical administration include lozenges comprising the active ingredient in a flavor, usually sucrose and acacia or tragacanth; pastilles comprising the active ingredient in an inert base, such as gelatin and glycerin, or sucrose and acacia; mouthwashes comprising the active ingredient in a suitable liquid carrier; or creams, emulsions, suspensions, solutions, gels, creams, pastes, foams, lubricants, sprays, suppositories, or the like.

A person skilled in the art will appreciate that a suitable or appropriate formulation can be selected, adapted or developed based upon the particular application at hand. In addition, the pharmaceutical compositions of the present invention may be prepared for administration by a variety of different routes, whether systemic, local, or both. Such examples, include, but are not limited to, administrations performed intraarticularly, intracranially, intradermally, intrahepatically, intramuscularly, intraocularly, intraperitoneally, intrathecally, intravenously, subcutaneously, transdermally, or directly into a atherosclerotic site, such as by direct injection, direct application, and/or by implanting a device into in an artery or other appropriate tissue site.

A hedgehog signaling modulating agent may be formulated to be contained within, or, adapted to release by a surgical or medical device or implant. In certain aspects, an implant may be coated or otherwise treated with an active hedgehog signaling agent. For example, hydrogels, or other polymers, such as biocompatible and/or biodegradable polymers, may be used to coat an implant with the compositions of the present invention (i.e., the composition may be adapted for use with a medical device by using a hydrogel or other polymer). Polymers and copolymers for coating medical devices with an agent are well-known in the art. Examples of implants include, but are not limited to, angioplasty balloons, stents, drug-eluting stents, sutures, prosthesis, vascular catheters, dialysis catheters, vascular grafts, prosthetic heart valves, cardiac pacemakers, implantable cardioverter defibrillators or IV needles. Merely by way of example, a stent or stent graft typically includes a slender fabric tubular graft portion and is normally used to reinforce or strengthen a weak spot in a body passageway, such as a blood vessel. Insertion of a stent graft may be performed by use of a catheter. Placement may be facilitated by balloon expansion, such as during or following a balloon angioplasty procedure, or, alternatively, the stent graft may be self expanding.

Dosages for hedgehog modulators of the invention can be in unit dosage form, such as a tablet or capsule. The term “unit dosage form,” as used herein, refers to physically discrete units suitable as unitary dosages for animal (e.g., human) subjects, each unit containing a predetermined quantity of an agent of the invention, alone or in combination with other therapeutic agents, calculated in an amount sufficient to produce the desired effect in association with a pharmaceutically acceptable diluent, carrier, or vehicle.

One skilled in the art can routinely determine the appropriate dose, schedule, and method of administration for the exact formulation of the composition being used, in order to achieve the desired effective amount or effective concentration of the agent in the individual patient. One skilled in the art also can readily determine and use an appropriate indicator of the “effective concentration” of the compounds of the present invention by a direct or indirect analysis of appropriate patient samples (e.g., blood and/or tissues), in addition to analyzing the appropriate clinical symptoms of the disease, disorder, or condition.

The dose of a hedgehog modulating recombinant protein or oxysterol, or composition thereof, administered to an animal, particularly a human, in the context of the present invention should be sufficient to effect at least a therapeutic response in the individual over a reasonable time frame. The exact amount of the dose will vary from subject to subject, depending on the species, age, weight and general condition of the subject, the severity or mechanism of any disorder being treated, the particular agent or vehicle used, its mode of administration and the like. The dose used to achieve a desired concentration in vivo will be determined by the potency of the particular oxysterol employed, the pharmacodynamics associated with the oxysterol in the host, with or without additional agents, the severity of the disease state of infected individuals, as well as, in the case of systemic administration, the body weight and age of the individual. The size of the dose may also be determined by the existence of any adverse side effects that may accompany the particular agent, or composition thereof, employed. It is generally desirable, whenever possible, to keep adverse side effects to a minimum.

For example, a dose can be administered in the range of from about 5 ng (nanograms) to about 1000 mg (milligrams), or from about 100 ng to about 600 mg, or from about 1 mg to about 500 mg, or from about 20 mg to about 400 mg. For example, the dose can be selected to achieve a dose to body weight ratio of from about 0.0001 mg/kg to about 1500 mg/kg, or from about 1 mg/kg to about 1000 mg/kg, or from about 5 mg/kg to about 150 mg/kg, or from about 20 mg/kg to about 100 mg/kg. For example, a dosage unit can be in the range of from about 1 ng to about 5000 mg, or from about 5 ng to about 1000 mg, or from about or from about 100 ng to about 600 mg, or from about 1 mg to about 500 mg, or from about 20 mg to about 400 mg, or from about 40 mg to about 200 mg of a compound of according to the present invention.

A dose can be administered once per day, twice per day, four times per day, or more than four times per day as required to elicit a desired therapeutic effect. For example, a dose administration regimen can be selected to achieve a blood serum concentration of a compound of the present invention in the range of from about 0.01 to about 1000 nM, or from about 0.1 to about 750 nM, or from about 1 to about 500 nM, or from about 20 to about 500 nM, or from about 100 to about 500 nM, or from about 200 to about 400 nM. For example, a dose administration regime can be selected to achieve an average blood serum concentration with a half maximum dose of a compound of the present invention in the range of from about 1 μg/L (microgram per liter) to about 2000 μg/L, or from about 2 μg/L to about 1000 μg/L, or from about 5 μg/L to about 500 μg/L, or from about 10 μg/L to about 400 μg/L, or from about 20 μg/L to about 200 μg/L, or from about 40 μg/L to about 100 μg/L.

A therapeutically effective dose of an oxysterol as described herein may include one which has a positive clinical effect on a patient as measured by the ability of the agent to improve atherosclerosis, or other related cardiovascular diseases or conditions. A therapeutically effective dose of an oxysterol may also include one which has a positive clinical effect on reducing the risk of developing atherosclerosis, or other related conditions. The therapeutically effective dose of each agent can be modulated to achieve the desired clinical effect, while minimizing negative side effects. The dosage of the agent may be selected for an individual patient depending upon the route of administration, severity of the disease, age and weight of the patient, other medications the patient is taking and other factors normally considered by an attending physician, when determining an individual regimen and dose level appropriate for a particular patient.

By way of example, the invention may include elevating endogenous, circulating oxysterol levels over the patient's basal level. In a normal adult levels are about 10-400 ng/ml depending on age and type of oxysterol, as measured by mass spectrometry. Those skilled in the art of pharmacology would be able to select a dose and monitor the same to determine if an increase circulating levels over basal levels has occurred.

When given in combined therapy, the other agent can be given at the same time as the hedgehog modulator, or the dosing can be staggered as desired. The two (or more) drugs also can be combined in a composition. Doses of each can be less when used in combination than when either is used alone. Certain embodiments may also include treatment with an additional agent which acts independently or synergistically with an oxysterol to improve vascular condition.

Recombinant hedgehog proteins and/or oxysterols and/or other hedgehog modulators may also be administered to cells and tissues and subjects at risk of atherosclerosis, in dosages and by routes effective to reduce, eliminate, prevent, or treat atherosclerotic lesions. Another embodiment of the invention is a kit useful for any of the methods disclosed herein, either in vitro or in vivo. Such a kit can comprise one or more of the oxysterols or pharmaceutical compositions discussed herein. Optionally, the kits comprise instructions for performing the method. Optional elements of a kit of the invention include suitable buffers, pharmaceutically acceptable carriers, or the like, containers, or packaging materials. The reagents of the kit may be in containers in which the reagents are stable, e.g., in lyophilized form or stabilized liquids. The reagents may also be in single use form, e.g., in single dosage form. A skilled worker will recognize components of kits suitable for carrying out any of the methods of the invention.

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments and the Examples provided below are to be construed as merely illustrative, and not limiting of the scope of the invention in any way whatsoever. The entire disclosure of all applications, patents, and publications cited herein are hereby incorporated by reference in their entirety.

### EXAMPLES

### Example 1

### Hedgehog Signaling is Activated in Mouse and Human Atherosclerotic Lesions

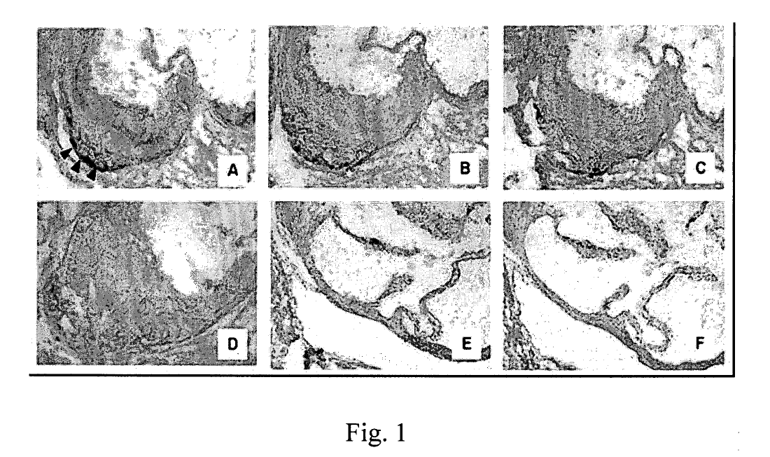

Immunohistochemical staining of lesions for diagnostic targets of Hh signaling, Ptch and Gli1, indicated that Hh signaling is highly activated in advanced atherosclerotic lesions from mice and humans. Frozen sections of mouse atherosclerotic lesions were stained with anti-Ptch antibody (FIG. 1A, FIG. 1D), anti-Gli1 antibody (FIG. 1B, FIG. 1E), anti-Shh antibody (FIG. 1F) or secondary antibody alone as negative control (FIG. 1C). Staining was developed using ABC and AEC kits from Vector Biolabs, and counterstaining was performed with hematoxylin.

Lesions obtained from apoEKO mice fed a high fat diet were stained for the above-described markers, and showed intense staining for Ptch in the shoulder regions and neointimal areas, associated with what appeared to be inflammatory cells and smooth muscle cells (FIG. 1A). In addition, Ptch staining was apparent around areas of calcification (typically staining intensely purple with hematoxylin in frozen specimens) and especially where an osteoid-like matrix appeared to be forming along the internal elastic lamina (IEL) (FIG. 1A, arrowheads). These sites are well known for being prominent areas associated with the initiation and progression of vascular calcification. Similar areas in serial sections were positively stained for Gli1, which is the transcription factor that is upregulated during Hh signaling and mediates the induction of Ptch and other Hh target gene expression (FIG. 1B). In addition, staining for Shh (FIG. 1D) was observed in similar areas of serial sections. In contrast, immunohistochemical examination of normal arteries from C57BLK/6 mice did not show staining for Ptch, Gli, (FIG. 1E, FIG. 1F) or Shh (data not shown).

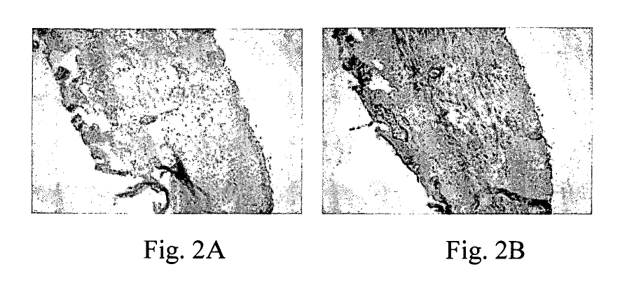

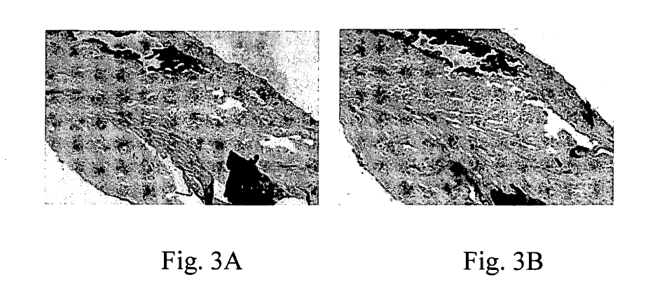

Intense Ptch staining was also observed in human atherectomy specimens from two human donors. Frozen sections of human atherosclerotic lesions were stained with anti-Ptch antibody (FIG. 2A) or secondary antibody alone as a negative control (FIG. 2B). In addition, frozen sections of human atherosclerotic lesions obtained from atherectomy were stained with anti Ptch antibody (FIG. 3A) or secondary antibody alone as a negative control (FIG. 3B). Staining was developed using ABC and AEC kits from Vector Biolabs, and counterstained with hematoxylin. Calcification appears as dark purple stained areas.

Ptch staining in these specimens was also associated with EC, areas of inflammatory cell infiltration, SMC proliferation, and vascular calcification (FIG. 2A, FIG. 3A). Not wishing to be bound by any theory, these findings are highly suggestive of the activation of Hh signaling in vascular cells during atherosclerosis and vascular calcification. The results provided herein identify the particular cells associated with increased Hh signaling in atherosclerotic lesions, to enable targeted treatment of such cells with hedgehog signaling modulators, such as oxysterols.

### Example 2

### Oxysterols Activate Hedgehog Signaling in Pluripotent Mesenchymal Cells

The inventors have previously demonstrated that oxysterols, including 22(R)-, 22(S)-, 20(S), and 25-hydroxycholesterol, possess potent osteoinductive and anti-adipogenic properties when applied to pluripotent mesenchymal cells (Kha et al., J Bone Miner Res 19:830-840 (2004)). The inventors have found that oxysterols activate Hh signaling in pluripotent mesenchymal cells, which appears to occur not through direct activation of Smo, but rather through a yet unidentified mechanism involving a target upstream of Smo.

According to the invention, oxysterols may also be used to activate Hh signaling in vascular mesenchymal cells (i.e., SMC/CVC) and EC. Similar to Shh-induced Hh signaling in vascular mesenchymal cells (as described herein), oxysterol-induced Hh signaling in these cells may modulate their inflammatory response to other atherogenic and/or osteoinductive factors, such as oxidized lipids and cytokines.

### Example 3

### Shh Modulates Oxidized Lipid-Induced IL-8 Expression in Aortic Endothelial Cells

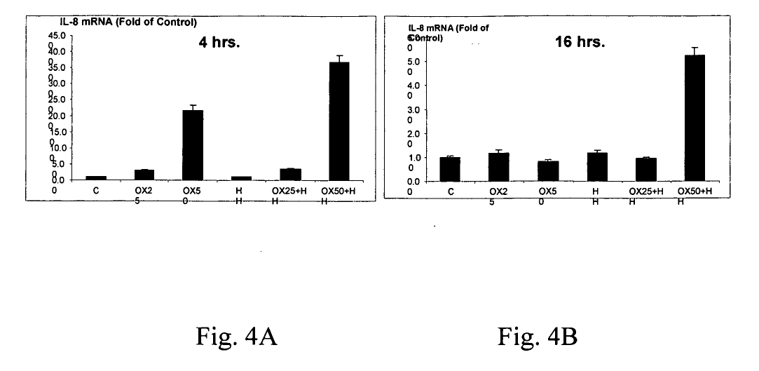

Activation of Hh signaling by Shh was examined to determine whether such signaling modulates inflammatory cytokine production in aortic endothelial cells (EC), similar to what has been reported in T cells. Primary human aortic EC (HAEC) were treated with Ox PAPC (Ox; (concentration shown on FIGS. 4A and 4B as μg/ml)) or Shh (1-1H, 200 ng/ml), alone or in combination as indicated in FIGS. 4A and 4B. After 4 and 16 hours of treatment, RNA was extracted from the cells and analyzed for IL8 mRNA expression by Q RT PCR and normalized to GAPDH expression.

Co-treatment of HAEC with Shh and oxidized palmitoyl-arachidonoyl-phosphocholine (Ox-PAPC), an oxidized phospholipid that has pro-inflammatory properties (see, e.g., Parhami et al., J Clin Invest 92:471-478 (1993); Vora et al., Circ Res 80:810-818 (1997)), caused a sustained activation of IL-8 mRNA expression in these cells after 16 hours of treatment (FIG. 4). At the 16 hour timepoint, Ox-PAPC-induced IL-8 expression (which is normally upregulated after 4 hours of treatment) returned to baseline levels in the absence of Shh, but remained elevated in the presence of Shh (FIG. 4). This finding suggests that induction of IL-8, and perhaps other inflammatory cytokines, by oxidized lipids can be modulated by the activation of Hh signaling. In quantitative reverse transcription polymerase chain reaction (Q-RT-PCR) studies, it was found that treatment of HAEC, as well as human microvascular endothelial cells (HMEC), for 8 hours with 400 ng/ml of rhShh caused a 2-fold increase in Ptch and Gli1 expression. Since EC may represent targets of Hh signaling, Shh and oxysterols that activate Hh signaling may regulate EC inflammatory responses, including cytokine production and binding to monocytes/macrophages. The origin and state of differentiation of the EC may determine their responsiveness to Shh (Shih et al., J Clin Invest 103:613-625 (1999)).

### Example 4

### Human Aortic Smooth Muscle Cells (HASMC) and Mouse Aortic Smooth Muscle Cells (MASMC) are Targets of Hedgehog Signaling

Treatment of HASMC with Shh was examined to determine if Shh induces the expression of target genes, Ptch and Gli. The levels of Shh required to activate the expression of these Hh target genes in HASMC, as well as in their mouse counterparts (MASMC), were observed to be much higher than the levels necessary for the activation of target genes in mouse bone marrow stromal cells. This difference may be cell-type specific or may be due to heterogeneity in vascular smooth muscle cell populations and their differentiation state. Ptch and Gli1 mRNA expression was induced when using 3 μg/ml of rmShh after 48 hours of treatment (1.6 fold and 1.8 fold, respectively, compared to control vehicle-treated cells as assessed by Q-RT-PCR analysis). This is consistent with studies in which 3.5 μg/ml of Shh was used to activate rat vascular SMC. It is likely that local Shh concentrations may reach very high levels in the microenvironment of the vessel wall, since, as described herein, substantial immunostaining for Shh in atherosclerotic lesions was observed. Cells within the atherosclerotic lesion may also become more susceptible to Shh over time due to the inflammatory insults associated with that environment. 20(S)-hydroxycholesterol also induced a 2-fold increase in Gli1 expression in HASMC and MASMC after 48 hours of treatment, suggesting that Hh signaling in these cells may be activated by Shh as well as by oxysterols. Dose response and time course experiments can determine the extent and kinetics of Hh target gene expression in aortic SMC, as well as in other vascular cells, using techniques known to a person of ordinary skill.

### Example 5

### Shh Protein Inhibits LPS-Mediated Macrophage Activation

Macrophage activation is a hallmark of atherosclerosis, and strategies to inhibit macrophage activation may have beneficial effects in intervention with atherosclerosis. Bacterial lipopolysaccharide (LPS) was used to analyze the effects of the hedgehog activation pathway on macrophage activation. Treatment of macrophages with Sonic hedgehog (Shh) protein was observed to inhibit the activation of these cells by lipopolysaccharide (LPS). Mouse peritoneal macrophages, which resemble circulating macrophages, were isolated from CD1 or C57BL/6 mice and treated in vitro with control vehicle (C), 100 pg/ml of LPS, or Shh (Shh was prepared in vitro in conditioned-medium from 293 cells transfected with a plasmid that produces Shh and used at 1:10 dilution), alone or in combination as indicated. After 4 hours, RNA was isolated and analyzed for the expression of tumor necrosis factor-α (TNF-α, a marker of inflammation and macrophage activation) by quantitative real time PCR. Data from a representative experiment are reported as the mean of triplicate determination±SD (p<0.001 for Control vs. LPS and for LPS vs. LPS+Shh).

This data indicates that activation of the hedgehog signaling pathway by any means, including, but not limited to, treatment with recombinant hedgehog proteins Shh, Indian hedgehog (Ihh), or Desert hedgehog (Dhh), in addition to oxysterols that activate the hedgehog pathway, or genetic manipulation to induce hedgehog protein expression, systemically or locally at the level of the artery wall, may inhibit macrophage activation and atherosclerosis in humans or animals.

The various embodiments described above can be combined to provide further embodiments. All of the U.S. patents, U.S. patent application publications, U.S. patent applications, U.S. provisional patent applications, foreign patents, foreign patent applications, and non-patent publications referred to in this specification, including but not limited to WO2004/019884, WO2005/020928, WO2006/110490, WO2007/028101, WO2007/098281, WO2008/011071, PCT/US2007/025833, U.S. Ser. Nos. 10/524,945, 10/569,994, 11/918,089, 11/991,322, 60/907,001, and 60/996,729 are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

### REFERENCES

1. Ross R. The pathogenesis of atherosclerosis: a perspective for the 1990s. Nature 362:801-809; 1993.
2. Badimon L, Martinez-Gonzalez J, Llorente-Cortes V, Rodriguez C, Padro T. Cell biology and lipoproteins in atherosclerosis. Curr Mol Med 6:439-456; 2006.
3. Libby P. Inflammation in atherosclerosis. Nature 420:868-874; 2002.
4. Shao J, Cai J, Towler D A. Molecular mechanisms of vascular calcification. Lessons learned from the aorta. Arterioscler Thromb Vasc Biol 26:1423-1430; 2006.
5. Demer L L, Tintut Y, Parhami F. Novel mechanisms of accelerated vascular calcification in renal disease patients. Curr Opin Nephrol Hyperten 11:437-443; 2002.
6. Parhami F, Morrow A D, Balucan J, Leitinger N, Watson A D, Tintut Y, Berliner J A, Demer L L. Lipid oxidation products have opposite effects on calcifying vascular cell and bone cell differentiation. A possible explanation for the paradox of arterial calcification in osteoporotic patients. Arterioscler Thromb Vasc Biol 17:680-687; 1997.
7. Tintut Y, Patel J, Parhami F, Demer L L. Tumor necrosis factor-alpha promotes in vitro calcification of vascular cells via cAMP pathway. Circulation 102:2636-2642; 2000.
8. Bijlsma M F, Peppelenbosch M P, Spek A. Hedgehog morphogen in cardiovascular disease. Circulation 114:1985-1991; 2006.
9. Morrow D, Sweeney C, Birney Y A, Guha S, Collins N, Cummins P M, Murphy R, Walls D, Redmond E M, Cahill P A. Biomechanical regulation of hedgehog signaling in vascular smooth muscle cells in vitro and in vivo. Am J Physiol Cell Physiol 2006 (in press).
10. Byrd N, Grabel L. Hedgehog signaling in murine vasculogenesis and angiogenesis. Trends Cardiovasc Med 14:308-313; 2004.
11. Stewart G A, Lowery J A, Wakelin S J, Fitch P M, Lindey S, Dallman M J, Lamb J R, Howie S E. Sonic hedgehog signaling modulates activation of and cytokine production by human peripheral CD4⁺ T cells. J Immunol 169:5451-5457; 2002.
12. Stewart G A, Hoyne G F, Ahmad S A, Jarman E, Wallace W A, Harrison D J, Haslett C, Lamb J R, Howie S E. Expression of the developmental Sonic hedgehog (Shh) signalling pathway is up-regulated in chronic lung fibrosis and the Shh receptor patched1 is present in circulating T lymphocytes. J Pathol 199:488-495; 2003.
13. Lees C, Howie S, Sartor R B, Satsangi J. The hedgehog signalling pathway in the gastrointestinal tract: implications for development, homeostasis, and disease. Gastroenterology 129:1696-1710; 2005.
14. Panakova D, Sprong H, Marois E, Thiele C, Eaton S. Lipoprotein particles are required for hedgehog and wingless signaling. Nature 435:58-65; 2005.
15. Lum L, Beachy P A. The hedgehog response network: sensors, switches, and routers. Science 304:1755-1759; 2004.
16. Bijlsma M F, Spek C A, Peppelenbosch M P. Hedgehog: an unusual signal transducer. BioEssays 26:387-394; 2004.
17. Spinella-Jaegle S, Rawadi G, Kawai S, Gallea S, Faucheu C, Mollat P, Courtois B, Bergaud B, Ramez V, Blanchet A M, Adelmant G, Baron R, Roman-Roman S. Sonic hedgehog increases the commitment of pluripotent mesenchymal cells into the osteoblastic lineage and abolishes adipocytic differentiation. Cell Sci 114:2085-2094; 2001.
18. Hilton M J, Tu X, Cook J, Hu H, Long F. Ihh controls cartilage development by antagonizing Gli3, but requires additional effectors to regulate osteoblast and vascular development. Development 132:4339-4351; 2005.
19. Chiang C, Litingtung Y, Lee E, Young K E, Corden J L, Westphal H, Beachy P A. Cyclopia and defective axial patterning in mice lacking sonic hedgehog gene function. Nature 383:407-413; 1996.
20. St-Jacques B, Hammerschmidt M, McMahon A P. Indian hedgehog signaling regulates proliferation and differentiation of chondrocytes and is essential for bone formation. Genes Develop 13:2072-2086; 1999.
21. Hu H, Hilton M J, Tu X, Yu K, Ornitz D M, Long F. Sequential roles of hedgehog and Wnt signaling in osteoblast development. Development 132:49-60; 2004.
22. Shao J, Cheng S, Pingsterhaus J M, Charlton-Kachigian N, Loewy A P, Towler D A. Msx2 promotes cardiovascular calcification by activating paracrine Wnt signals. J Clin Invest 115:1210-1220.
23. Edwards P A, Ericsson J. Sterols and isoprenoids: signaling molecules derived from the cholesterol biosynthetic pathway. Annu Rev Biochem 68:157-185; 1999.
24. Bjorkhem I, Meaney S, Diczfalusy U. Oxysterols in human circulation: which role do they play? Curr Opion Lipidol 13:247-253; 2002.
25. Olkkonen V M, Lehto M. Oxysterols and oxysterol binding proteins: role in lipid metabolism and atherosclerosis. Ann Med 36:562-572; 2004.
26. Bjorkhem I, Diczfalusy U. Oxysterols: friends, foes, or just fellow passengers? Arterioscler Thromb Vasc Biol 22:734-742; 2002.
27. Zelcer N, Tontonz P. Liver X receptors as integrators of metabolic and inflammatory signaling. J Clin Invest 116:607-614; 2006.
28. Kha H T, Basseri B, Shouhed D, Richardson J, Tetradis S, Hahn T J, Parhami F. Oxysterols regulate differentiation of mesenchymal stem cells: pro-bone and anti-fat. J Bone Miner Res 19:830-840; 2004.
29. Dwyer J R, Sever N, Carlson M, Nelson S F, Beachy P A, Parhami F. Oxysterols are novel activators of the hedgehog signaling pathway in pluripotent mesenchymal cells. J Biol Chem 2007 (in press).
30. Shouhed D, Kha H T, Richardson J A, Amantea C M, Hahn T J, Parhami F. Osteogenic oxysterols inhibit the adverse effects of oxidative stress on osteogenic differentiation of marrow stromal cells. J Cell Biochem 95:1276-1283; 2005.
31. Watson K E, Bostrom K, Ravindranath R, Lam T, Norton B, Demer L L. TGF-beta and 25-hydroxycholesterol stimulate osteoblast-like vascular cells to calcify. J Clin Invest 93:2106-2113; 1994.
32. Berliner J A, Watson A D. A role for oxidized phospholipids in atherosclerosis. N Engl J Med 353: 9-12; 2005.
33. Watson A D, Subbanagounder G, Welsbie D S, Faull K F, Navab M, Jung M E, Fogelman A M, Berliner J A. Structural identification of a novel pro-inflammatory epoxyisoprostane phospholipid in mildly oxidized low density lipoprotein. J Biol Chem 274:24787-24798; 1999.
34. Hochman E, Castiel A, Jacob-Hirsch J, Amariglio N, Izraeli S. Molecular pathways regulating pro-migratory effects of hedgehog signaling. J Biol Chem 281:33860-33870; 2006.
35. Parhami F, Tintut Y, Ballard A, Fogelman A M, Demer L L. Leptin enhances the calcification of vascular cells: artery wall as a target of leptin. Circ Res 88:954-960; 2001.
36. Speer M Y, Chien Y, Quan M, Yang H, Vali H, McKee M D, Giachelli C M. Smooth muscle cells deficient in osteopontin have enhanced susceptibility to calcification in vitro. Cardiovasc Res 66:324-333; 2005.
37. Bidder M, Shao J, Charlton-Kachigian N, Loewy A P, Semenkovich C F, Towler D A. Osteopontin transcription in aortic smooth muscle cells is controlled by glucose-regulated upstream stimulatory factor and activator protein-1 activities. J Biol Chem 277:44485-44496; 2002.
38. Stein G S, Lian J B. Molecular mechanisms mediating proliferation/differentiation interrelationships during progressive development of the osteoblast phenotype. Endocrine Rev 14:424-442; 1993.
39. Parhami F, Fang Z T, Fogelman A M, Andalibi A, Territo M C, Berliner J A. Minimally modified low density lipoprotein-induced inflammatory responses in endothelial cells are mediated by cyclic adenosine monophosphate. J Clin Invest 92:471-478; 1993.
40. Vora D K, Fang Z T, Liva S M, Tyner T R, Parhami F, Watson A D, Drake T A, Territo M C, Berliner J A. Induction of P-selectin by oxidized lipoproteins. Separate effects on synthesis and surface expression. Circ Res 80:810-818; 1997.
41. Shih P T, Elices M J, Fang Z T, Ugarova T P, Strahl D, Territo M C, Frank J S, Kovach N L, Cabanas C, Berliner J A, Vora D K. Minimally modified low-density lipoprotein induces monocyte adhesion to endothelial connecting segment-1 by activating beta1 integrin. J Clin Invest 103:613-625; 1999.
42. Braunersreuther V, Mach F. Leukocyte recruitment in atherosclerosis: potential targets for therapeutic approaches? Cell Mol Life Sci 63:2079-2088; 2006.
43. Bostrom K, Watson K E, Horn S, Wortham C, Herman I M, Demer L L. Bone morphogenetic protein expression in human atherosclerotic lesions. J Clin Invest 91:1800-1809; 1993.
44. Shin V, Zebboudj A F, Bostrom K. Endothelial cells modulate osteogenesis of calcifying vascular cells. J Vasc Res 41:193-201; 2004.
45. Dijke P, Korchynskyi O, Valdimarsdottir G, Goumans M. Controlling cell fate by bone morphogenetic protein receptors. Mol Cell Endocrinol 211:105-113; 2003.
46. Zhao M, Qiao M, Harris S E, Chen D, Oyajobi B O, Mundy G R. The zinc finger transcription factor Gli2 mediates bone morphogenetic protein 2 expression in osteoblasts in response to hedgehog signaling. Mol Cell Biol 26:6197-6208; 2006.
47. Yeh M, Gharavi N M, Choi J, Hsieh X, Reed E, Mouillesseaux K P, Cole A L, Reddi S T, Berliner J A. Oxidized phospholipids increase interleukin 8 (IL-8) synthesis by activation of the c-src/signal transducers and activators of transcription (STAT)₃ pathway. J Biol Chem 279:30175-30181; 2004.

1. A method of treating a cardiovascular disease, comprising administering to a subject having or at risk of having the cardiovascular disease a therapeutically effective amount of a composition comprising a modulator of hedgehog activity, wherein the composition comprises (a) a recombinant hedgehog protein, or a biologically active fragment or variant of a recombinant hedgehog protein, and (b) an oxysterol.

2-16. (canceled)

17. The method of claim 1, wherein the cardiovascular disease is selected from the group consisting of atherosclerosis, vascular calcification, aneurysms, angina, arrhythmia, cardiomyopathy, stroke, cerebrovascular disease, chronic inflammatory disease, congenital heart disease, congestive heart failure, coronary artery disease, myocarditis, valve disease, dilated cardiomyopathy, diastolic dysfunction, endocarditis, gangrene, hypertension, hypertrophic cardiomyopathy, ischemic heart disease, inflammatory heart disease, macrophage activation syndrome, mitral valve prolapse, myocarditis, myocardial infarction, venous thromboembolism, peripheral artery occlusive disease, stenosis, and restenosis.

18. A method for treating atherosclerosis, comprising the step of administering to a subject having or at risk for having atherosclerosis a therapeutically effective amount of a composition comprising (a) an agent selected from the group consisting of a recombinant hedgehog protein, and a biologically active fragment or variant of a recombinant hedgehog protein, and (b) an oxysterol, thereby treating atherosclerosis.

19. A method of reducing the pathogenesis of atherosclerosis, comprising the step of contacting vascular cells, immune cells, or stem cells with (a) a recombinant hedgehog protein, or a biologically active fragment or variant of a recombinant hedgehog protein, and (b) an oxysterol, in an amount effective to modulate hedgehog signaling in the cells, thereby reducing the pathogenesis of atherosclerosis.

20. The method of claim 19, wherein:
the vascular cells are selected from the group consisting of mature endothelial cells, progenitor endothelial cells, vascular smooth muscle cells, pericytes, adipocytes, calcifying vascular cells, myofibroblasts, and pluripotent mesenchymal cells;
the immune cells are selected from the group consisting of monocytes, macrophages, T cells, B cells, and dendritic cells;
the stem cells are selected from the group consisting of mesenchymal stem cells, bone marrow stromal cells, and hematopoietic stem cells.

21.-24. (canceled)

25. A method of reducing the pathogenesis of vascular calcification, comprising the step of contacting vascular or immune cells with (a) a recombinant hedgehog protein, or a biologically active fragment or variant of a recombinant hedgehog protein, and (b) an oxysterol, in an amount effective to modulate hedgehog signaling in the cells, thereby reducing the pathogenesis of vascular calcification.

26. (canceled)

27. The method of claim 1, wherein the recombinant hedgehog protein, or a biologically active fragment or variant thereof, is selected from the group consisting of Sonic hedgehog (Shh), Indian hedgehog (Ihh) and Desert hedgehog (Dhh) proteins.

28. The method of claim 1, wherein the oxysterol is selected from the group consisting of:
(i) 22(S)-hydroxycholesterol, 22(R)-hydroxycholesterol, 20(S)-hydroxycholesterol, 5-cholesten-3beta, 20alpha-diol 3-acetate, 24-hydroxycholesterol, 24(S), 25-epoxycholesterol, pregnenolone, 26-hydroxycholesterol, and 4beta-hydroxycholesterol;
(ii) Oxy3, Oxy4, Oxy7, Oxy8, Oxy9, Oxy10, or Oxy11;
(iii) Oxy12, Oxy13, Oxy14, Oxy15, Oxy20, Oxy22, Oxy26, Oxy27, Oxy28, Oxy34, Oxy36, Oxy38, Oxy39, Oxy 40, Oxy41, Oxy42, Oxy48, or Oxy49;
(iv) 24S-hydroxycholesterol or 25-hydroxycholesterol;
(v) Oxy6; and
(vi) Oxy5.

29.-37. (canceled)

38. The method of claim 28, wherein the composition is administered via an implant device.

39. The method of claim 38, wherein the implant device is selected from the group consisting of angioplasty balloons, stents, drug-eluting stents, sutures, prosthesis, vascular catheters, dialysis catheters, vascular grafts, prosthetic heart valves, cardiac pacemakers, implantable cardioverter defibrillators, and IV needles.

40. The method of claim 28, wherein the composition is adapted for coating onto a medical device.

41. A method comprising modulating hedgehog signaling in target cells involved in the pathogenesis of atherosclerosis and/or vascular calcification, comprising contacting the target cells with (a) a recombinant hedgehog protein, or a biologically active fragment or variant of a recombinant hedgehog protein, and (b) an oxysterol, in an amount effective to modulate hedgehog signaling in the cells and thereby to treating atherosclerosis or its cellular processes.

42. The method of claim 41, wherein the target cells are in culture.

43. The method of claim 41, wherein the target cells are cells of a subject, human, or animal.

44. (canceled)

45. A method of treating a cardiovascular disease, comprising administering to a subject having or at risk of having the cardiovascular disease a therapeutically effective amount of a composition comprising a modulator of hedgehog activity, wherein the modulator is selected from the group consisting of Oxy1, Oxy2, and Oxy16.

46. A composition comprising one or more of the following oxysterol compounds: Oxy20, Oxy22, Oxy26, Oxy27, Oxy28, Oxy34, Oxy38, Oxy39, Oxy40, Oxy41, Oxy48, or Oxy49.
